(19) United States
(12) Patent Application Publication
(10) Pub. No.: US 2026/0240885 A1
(43) Pub. Date: Aug. 20, 2026

(54) PHOSPHORYLATED HEXAACYL DISACCHARIDES FOR TREATING OR PREVENTING ACUTE KIDNEY INJURY

(71) Applicant: VANDERBILT UNIVERSITY, Nashville, TN (US)

(72) Inventor: Antonio HERNANDEZ, Nashville, TN (US)

(21) Appl. No.: 19/130,654

(22) PCT Filed: Nov. 17, 2023
(86) PCT No.: PCT/US2023/080279

§ 371 Date: May 16, 2025

Related U.S. Application Data
(60) Provisional application No. 63/384,269, filed on Nov. 18, 2022.

(51) Int. Cl. A61K 31/7028; A61K 9/00; A61P 13/12

(52) U.S. Cl. CPC A61K 31/7028; A61K 9/0019; A61P 13/12

(57) ABSTRACT

The present disclosure relates to phosphorylated hexa-acyl disaccharide (PHAD) compounds and methods for preventing ischemic injuries.

### CROSS-REFERENCE TO RELATED APPLICATIONS

This PCT application claims priority to, and the benefit of, U.S. Provisional Patent Application No. 63/384,269, filed Nov. 18, 2022, which is incorporated by reference herein in its entirety.

### STATEMENT REGARDING FEDERALLY SPONSORED RESEARCH

This invention was made with government support under Grant No. K08 GM123345 awarded by the National Institutes of Health. The government has certain rights in the invention.

### REFERENCE TO SEQUENCE LISTING

The sequence listing submitted on Nov. 17, 2023, as an .XML file entitled “10644-155WO1_ST26.xml” created on Nov. 15, 2023, and having a file size of 26,143 bytes is hereby incorporated by reference pursuant to 37 C.F.R. § 1.52(e)(5).

### FIELD

The present disclosure relates to phosphorylated hexa-acyl disaccharide (PHAD) compounds and methods for preventing ischemic injuries.

### BACKGROUND

Surgical and critically ill patient populations have a higher predisposition of developing acute kidney injury (AKI). Ischemia reperfusion induced acute kidney injury (IRI-AKI) contributes to the pathogenesis of acute kidney injury after a variety of major surgical procedures. In non-cardiac surgery patients, the incidence of AKI is as high as 12% and is associated with increased morbidity to include development of chronic kidney disease and mortality. Patients undergoing cardiac surgery have a higher incidence of AKI ranging from 7% to 40%, with up to 3% developing severe, dialysis dependent AKI. There are no definitive FDA approved therapies to prevent AKI after surgery, while alternative strategies, such as remote ischemic preconditioning, have shown mixed results. Thus, there is an unmet need for the prevention of AKI in perioperative medicine.

The compounds and methods disclosed herein address these and other needs.

### SUMMARY

The present disclosure also provides methods using compounds to treat or prevent ischemic injuries during surgery.

In one aspect, disclosed herein is a method of treating or preventing an acute kidney injury, comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

In one aspect, disclosed herein is a method of preserving kidney function during a surgical procedure, the method comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

In one aspect, disclosed herein is a method of treating or preventing an ischemic injury during a surgical procedure, the method comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

In one embodiment, the compound is a phosphorylated hexa-acyl disaccharide (PHAD). In another embodiment, the compound is a 3-deacyl phosphorylated hexa-acyl disaccharide (3D-PHAD). In another embodiment, the compound is a 3-D (6-acyl) phosphorylated hexa-acyl disaccharide (3D(6-acyl)PHAD).

In some embodiments, the compound is a synthetic toll-like receptor 4 (TLR4) agonist. In some embodiments, the compound is administered intravenously. In some embodiments, the compound is administered at a dose between 0.5 mg/kg-40 mg/kg. In some embodiments, the compound is administered about 24 hours or less prior to a surgical procedure. In some embodiments, the compound protects one or both kidneys from damage during the surgical procedure.

In another embodiment, the surgical procedure is selected from the group consisting of a cardiac surgery, a vascular surgery, a neurosurgery, a genitourinary surgery, an abdominal surgery, and a kidney surgery. In some embodiments, the acute kidney injury is an ischemia reperfusion-induced acute kidney injury (IRI-AKI). In some embodiments, the compound is further administered with an antibiotic, a laxative, an anesthetic, a muscle relaxant, a sedative, or any combination thereof prior to the surgical procedure.

In another embodiment, the subject is a mammal. In one embodiment, the subject is a human.

### BRIEF DESCRIPTION OF FIGURES

The accompanying figures, which are incorporated in and constitute a part of this specification, illustrate several aspects described below.

FIGS. 1A, 1B, 1C, 1D, and 1E show the pretreatment with PHAD in mice undergoing unilateral IRI-AKI. Mice were pretreated with intravenous PHAD at 2, 20 and 200 μg/mouse or vehicle control, 48 and 24 hours prior to undergoing right nephrectomy followed by clamping of the left renal pedicle for 28 minutes. FIG. 1A shows that blood was analyzed for BUN and creatinine at baseline (0), and post-injury day 1 and 3. Results expressed as means+/−SEM with N=8. Two-way ANOVA was used to compare differences between PHAD- and vehicle-treated mice over time, with p values indicated. FIG. 1B shows the representative images of PAS-stained sections of the outer medulla Day 3 after injury in sham, vehicle- and PHAD-treated mice. Arrows point to casts within the collecting tubules. Scale bar, 100 μM. FIG. 1C shows the median tubular injury scores in the outer stripe of the outer medulla from PAS-stained sections Day 3 after injury in sham, vehicle and PHAD-treated mice. FIG. 1D shows the 3-day survival curves. Group differences were compared by log-rank test (p>0.05). FIG. 1E shows the median qRT-PCR for renal expression of N-Gal, Kim-1, IL-6, Tnf-α, and IL-1β mRNAs. GAPDH was used as the internal control for qRT-PCR, and ΔCt values graphed are relative to the sham group. For FIGS. 1C and 1D, individual data points and medians are shown, and between group differences compared by one way ANOVA, using Dunnett's post hoc correction for multiple between group comparison Kruskal-Wallis test, with significant p values (<0.05), as indicated. N=6.

FIGS. 2A, 2B, 2C, 2D, 2E, and 2F show the pretreatment with PHAD in mice undergoing bilateral IRI-AKI. Mice were pretreated with intravenous PHAD at 200 μg/mouse or vehicle control, 48 and 24 hours prior to undergoing bilateral renal pedicle clamping for 24 minutes. FIG. 2A shows the blood was analyzed for BUN and creatinine at baseline (0), and post-injury day 1 and 3. Results expressed as means+/−SEM with N=10. Two-way ANOVA was used to evaluate between group differences over time (p<0.05 for both BUN and serum creatinine), with p values shown after Sidak's correction for multiple post hoc between group comparisons at each time point. FIG. 2B shows the tubular injury scores in the outer stripe of the outer medulla from PAS-stained sections Day 3 after injury. FIG. 2C shows the Apoptosis in the outer stripe of the outer medulla from TUNEL stained sections Day 3 after injury. FIG. 2D shows the representative images of PAS-stained sections of the outer medulla Day 3 after injury in sham, vehicle and PHAD-treated mice. Arrows point to tubular casts. FIG. 2E shows the representative images of TUNEL stained sections of the outer medulla day 3 after injury in vehicle and PHAD-treated mice. Red arrow heads indicate examples of TUNEL+ tubular nuclei, and yellow dotted lines indicate necrotic TUNEL positive tubular casts excluded from the analysis. FIG. 2F shows the qRT-PCR for renal expression of N-Gal, Kim-1, IL-6, Tnf-α, and IL-1β mRNAs Day 3 after injury. GAPDH was used as the internal control for qRT-PCR, and ΔCt values graphed are relative to the vehicle group. FIGS. 2B, 2C, and 2F show the individual data points and medians shown, and p values when significant difference are present are shown from a Mann-Whitney U test used to compare groups. N=6-7.

FIGS. 3A and 3B show the immunofluorescence staining for Kim-1 and F4/80 after bilateral IRI-AKI in mice pretreated with PHAD. Mice were pretreated with intravenous PHAD at 200 μg/mouse or vehicle control, 48 and 24 hours prior to undergoing bilateral renal pedicle clamping for 24 minutes. FIG. 3A show the representative images stained for expression of Kim-1, expressed by injured proximal tubules (left panels), and F4/80 expressed by renal macrophages (right panels) in sequential sections both co-labeled with LTL to mark proximal tubular cells in vehicle vs. PHAD treated mice 3 days after injury. White dotted lines indicate junction between the cortex and outer medulla which is only seen in the upper panels. Scale bars 50 microns. FIG. 3B show the quantification of Kim-1 and F4/80 positive areas in the cortex and outer stripe of the outer medulla in vehicle and PHAD treated mice at Day 3 after injury. Individual data points and medians shown, and p values shown from a Mann-Whitney U test used to compare groups, N=8.

FIGS. 4A, 4B, 4C, and 4D show the pretreatment with intravenous PHAD in mice undergoing bilateral IRI-AKI. Mice were pretreated with intravenous PHAD at 200 μg/mouse or vehicle control, 48 and 24 hours prior to undergoing bilateral renal pedicle clamping for 24 minutes. FIGS. 4A, 4B, 4C, and 4D show the qRT-PCR for renal expression of Ho-1 (FIG. 4A), Irf-1 (FIG. 4B), Ccl-2 (FIG. 4C), and Ccl-3 (FIG. 4D), Day 3 after injury. GAPDH was used as the internal control for qRT-PCR, and ΔCt values graphed are relative to the vehicle group. Individual data points and medians shown, and p values shown from Mann-Whitney U test used to compare groups, N=6-7.

FIG. 5 shows the validation of immunofluorescence staining with Kim-1 and F4/80 antibodies. Kidney sections from uninjured mice and mice 3 days after bilateral IRI-AKI were prepared and co-labeled with LTL and either rat anti-Kim-1 monoclonal antibody (R&D systems, MAB1817), or rat anti-F4/80 monoclonal antibody diluted to 1/250 [Cl:A3-1](abcam, ab6640), as described in the Materials and Methods of Example 2. Scale bar—50 μm.

FIG. 6 shows the survival in mice pretreated with PHAD undergoing bilateral IRI-AKI. Mice were pretreated with intravenous PHAD at 200 μg/mouse or vehicle control, 48 hours and 24 hours prior to undergoing bilateral renal pedicle clamping for 24 minutes. 3-day survival curves. Group differences were compared by log-rank test (p>0.05).

FIG. 7 shows a summary of the present disclosure, wherein pre-treatment of with a Toll-like receptor 4 agonist attenuates IRI-AKI.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100%, or more, increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” means lowering of an event or characteristic (e.g., kidney injury). It is understood that this is typically in relation to some standard or expected value, in other words it is relative, but that it is not always necessary for the standard or relative value to be referred to. For example, “reduces kidney injury” means reducing any physical damage caused to kidney tissue during a surgery relative to a standard or a control.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Prophylactic administration can occur for several days to years prior to the manifestation of symptoms of an infection.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

An “agonist” refers to a chemical composition or compound that activates a receptor protein to produce a biological response.

A “surgery” or a “surgical procedure” refers to a medical operation using specified techniques on a subject to investigate or treating a pathological condition such as a disease or an injury to help improve bodily functions, appearance, or to repair unwanted ruptured areas.

As used herein, “ischemia” refers to an inadequate blood supply to an organ or part of the body. Ischemia can occur to any tissue or organ that requires or has an established blood supply, including but not limited to the heart, liver, kidneys, brain, and muscles.

“Ischemia-reperfusion” refers to an occurrence when cellular dysfunction and death follow restoration of blood flow to a tissue or organ that previously experienced ischemia.

### Methods of Treatment

Acute kidney injury (AKI) is a condition in which one or both kidneys stop working properly, and ranges from minor loss of function to complete kidney failure. Normal functions of the kidneys include waste, drug, and toxin removal from the body, balance of bodily fluids, hormonal regulation of blood pressure, vitamin D production, and regulation of red blood cell production. Disruption to any one or a combination of these functions for about 7 days leads to AKI. AKI can indirectly result from other serious diseases, illness, or conditions, such as complications from surgical procedures and can also result from direct injury to the kidneys. There are currently no approved therapies or treatments for preventing or treating AKI after surgery.

AKI is a complication of cardiac and non-cardiac surgeries, which if left untreated for longer than 1, 2, 3, 4, 5, 6, or 7 days can lead to chronic kidney disease or chronic kidney failure. Subjects with underlying renal complications are at higher risk of developing AKI or chronic kidney disease following surgery. AKI during or following surgery mainly occurs from renal hypoperfusion (or decrease blood pressure in the kidneys) or renal inflammation. The AKI inflammation is caused by blood flow obstruction (ischemia) or instabilities, renal toxicity from antibiotics and other drugs given prior to surgery, or release of signaling molecules from damaged or dying renal cells. Another cause of AKI is ischemia-reperfusion injury (also referred to as ischemia reperfusion injury—acute kidney injury (IRI-AKI)) caused by restoration of blood supply after a period of ischemia. Therefore, there is a need for a treatment to prevent AKI during surgeries.

Therefore, the present disclosure provides methods using compounds to treat or prevent ischemic injuries during surgery. The present disclosure also relates to using PHAD compounds to prevent and treat perioperative injuries. PHADs are found to be agonists of toll-like receptors (TLRs), specifically they are TLR4 agonists functioning within innate immunity and modulating inflammation.

In one aspect, disclosed herein is a method of treating or preventing an acute kidney injury, comprising administering to a subject in need thereof a pharmaceutically effective amount of a compound selected from a phosphorylated hexa-acyl disaccharide (PHAD, Compound 1), a 3-deacyl phosphorylated hexa-acyl disaccharide (3D-PHAD, Compound 2), a 3-D (6-acyl) phosphorylated hexa-acyl disaccharide (3D(6-acyl)PHAD, Compound 3), or a pharmaceutically acceptable salt thereof.

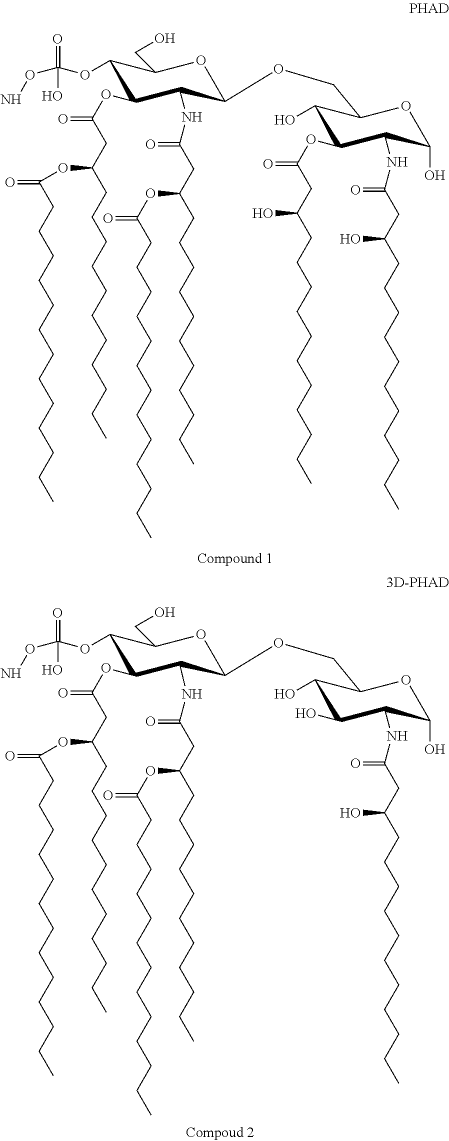

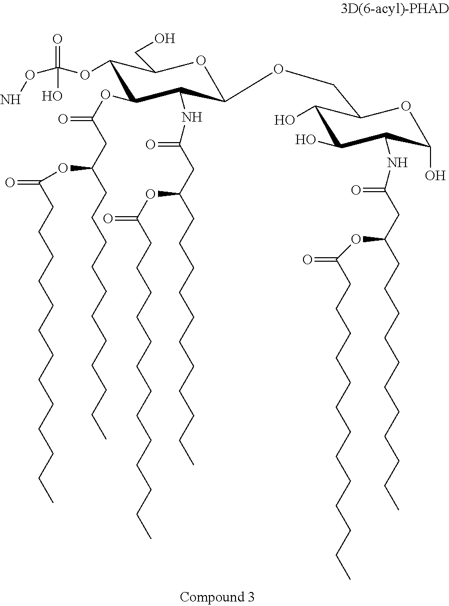

In one aspect, disclosed herein is a method of preserving kidney function during a surgical procedure, the method comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

In one aspect, disclosed herein is a method of treating or preventing an ischemic injury during a surgical procedure, the method comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

The condition of limited or complete loss of blood supply to a tissue, organ, or organ system, often referred to as ischemia, often leads to systemic or local organ dysfunction. Also, ischemia within one tissue, organ, or organ system can directly or indirectly impact other surrounding or distant tissues, organs, or organ systems. Major surgeries, such as cardiac surgeries and non-cardiac surgeries, can lead to ischemia of tissue, organ, organ system, or combinations thereof. However, preventive treatments of ischemia following surgery is lacking. Therefore, there is a need for treatments methods to prevent ischemic injury during a surgical procedure.

In one aspect, disclosed herein is a method of protecting a subject against cardiac, brain, or liver ischemia reperfusion injury, comprising administering to a subject in need thereof a pharmaceutically effective amount of a compound selected from a phosphorylated hexa-acyl disaccharide (PHAD, Compound 1), a 3-deacyl phosphorylated hexa-acyl disaccharide (3D-PHAD, Compound 2), a 3-D (6-acyl) phosphorylated hexa-acyl disaccharide (3D(6-acyl)PHAD, Compound 3), or a pharmaceutically acceptable salt thereof.

In one embodiment, the compound is a phosphorylated hexa-acyl disaccharide (PHAD). In another embodiment, the compound is a 3-deacyl phosphorylated hexa-acyl disaccharide (3D-PHAD). In another embodiment, the compound is a 3-D (6-acyl) phosphorylated hexa-acyl disaccharide (3D(6-acyl)PHAD). In one embodiment, the compound is a synthetic toll-like receptor 4 (TLR4) agonist.

The PHAD compound of any preceding aspect may be administered in such amounts, time, and route deemed necessary in order to achieve the desired result. The exact amount of the PHAD compound of any preceding aspect will vary from subject to subject, depending on the species, age, and general condition of the subject, the severity of the kidney injury/damage, the particular PHAD compound, its mode of administration, its mode of activity, and the like. The PHAD compound of any preceding aspect is preferably formulated in dosage unit form for ease of administration and uniformity of dosage. It will be understood, however, that the total daily usage of the PHAD compound of any preceding aspect will be decided by the attending physician within the scope of sound medical judgment. The specific therapeutically effective dose level for any particular subject will depend upon a variety of factors including the IRI injury being treated and the severity of the injury or damage; the activity of the PHAD compound of any preceding aspect employed; the specific PHAD compound of any preceding aspect employed; the age, body weight, general health, sex and diet of the patient; the time of administration, route of administration, and rate of excretion of the specific PHAD compound of any preceding aspect employed; the duration of the treatment; drugs used in combination or coincidental with the specific PHAD compound of any preceding aspect employed; and like factors well known in the medical arts.

The PHAD compound of any preceding aspect may be administered by any route. In some embodiments, the PHAD compound of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal. In some embodiments, the compound is administered intravenously. In some embodiments, the compound is administered by injection or instillation. In general, the most appropriate route of administration will depend upon a variety of factors including the nature of the PHAD compound of any preceding aspect (e.g., its stability in the environment of the subject's body), the condition of the subject (e.g., whether the subject is able to tolerate administration), etc.

The exact amount of PHAD compound of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

The concentration of active agent(s) can vary widely and will be selected primarily based on activity of the active ingredient(s), body weight and the like in accordance with the particular mode of administration selected and the patient's needs. In some embodiments, the compound is administered at a dose between 0.5 mg/kg-40 mg/kg. In some embodiments, compound is administered at a dose of about 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mg/kg.

Concentrations, however, will typically be selected to provide dosages ranging from about 0.5 mg/kg to about 40 mg/kg and sometimes higher. Non-limiting dose ranges include about 0.5 mg/kg/day to about 40 mg/kg/day, 0.75 mg/kg/day to about 40 mg/kg/day, 1 mg/kg/day to about 40 mg/kg/day, 5 mg/kg/day to about 40 mg/kg/day, 10 mg/kg/day to about 40 mg/kg/day, 15 mg/kg/day to about 40 mg/kg/day, 20 mg/kg/day to about 40 mg/kg/day, 25 mg/kg/day to about 40 mg/kg/day, 30 mg/kg/day to about 40 mg/kg/day, and 35 mg/kg/day to about 40 mg/kg/day. It should be understood that the dosage range can be increased, decreased, or any dosage in between the ranges disclosed above. It will be appreciated that such dosages may be varied to optimize a therapeutic regimen in a particular subject or group of subjects.

In one aspect, disclosed herein is a PHAD compound of any preceding aspect and a pharmaceutically acceptable carrier selected from an excipient, a diluent, a salt, a buffer, a stabilizer, a lipid, an emulsion, and a nanoparticle. One or more active agents (e.g. the PHAD) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the PHAD compound of any preceding aspect can be prepared as a “concentrate”, e.g. in a storage container of a premeasure volume and/or a predetermined amount ready for dilution, or in a soluble capsule ready for addition to a specified volume of water, saline, alcohol, hydrogen peroxide, or other diluent.

In some embodiments, the PHAD compound of any preceding aspect is administered about 24 hours or less prior to a surgical procedure. In some embodiments, the compound is administered about 0.5, 1, 2, 3, 4, 5, 6, 7, 8, 9, 10, 11, 12, 13, 14, 15, 16, 17, 18, 19, 20, 21, 22, 23, or 24 hours prior to a surgical procedure. In some embodiments, the PHAD compound of any preceding aspect is administered 1, 2, 3, 4, 5, 6, 7, 8, 9, 10, or more times prior to a surgical procedure. In some embodiments, the compound protects one or both kidneys from damage during the surgical procedure.

In some embodiments, the surgical procedure is selected from the group consisting of a cardiac surgery, a vascular surgery, a neurosurgery, a genitourinary surgery, an abdominal surgery, and a kidney surgery. In some embodiments, the acute kidney injury is an ischemia reperfusion-induced acute kidney injury (IRI-AKI).

In some embodiments, the compound is further administered with an antibiotic, a laxative, an anesthetic, a muscle relaxant, a sedative, or any combination thereof prior to the surgical procedure. In some embodiments, the compound is further administered with an antibiotic, such as those including but not limited to cefazolin, vancomycin, neomycin, erythromycin, and gentamicin. In some embodiments, the compound is further administered with a laxative, such as those including but not limited to magnesium-based laxatives. In some embodiments, the compound is further administered with an anesthetic, a muscle relaxant, or sedative, such as those including but not limited to propofol, etomidate, ketamine, procaine, benzocaine, tetracaine, lidocaine, prilocaine, levobupivacaine, bupivacaine, dibucaine, isoflurane, desflurane, sevoflurane, amobarbital, methohexital, thiamylal, thiopental, diazepam, lorazepam, midazolam, alfentanil, fentanyl, remifentanil, succinylcholine (suxamethonium), decamethonium, mivacurium, rapacuronium, atracurium, cisatracurium, rocuronium, gallamine, alcuronium, doxacurium, metocurine, pancuronium, pipecuronium, and tubocurarine.

In some embodiments, the subject is a mammal. In some embodiments, the subject is a dog, a cat, a mouse, a rabbit, a cow, a horse, a sheep, or a non-human primate.

In some embodiments, the subject is a human.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present disclosure which are apparent to one skilled in the art.

### Example 1: Acute Kidney Injury (AKI) is a Common Occurrence in Surgical and Critically Ill Patients

This example shows that induction of trained immunity attenuates ischemia reperfusion-induced AKI (IRI-AKI).

A blinded, randomized controlled study in mice, assessed the efficacy of intravenous 3D 6-Acyl phosphorylated hexa-acyl disaccharide (PHAD, a de novo ultrapure synthetic toll-like receptor 4 agonist), to protect against IRI-AKI. Two cohorts of male BALB/c mice received intraperitoneal vehicle or PHAD (10, 20, or 40 μg); or intravenous vehicle or PHAD (2, 20, or 200 μg) at 48 and 24 hours prior to unilateral renal pedicle clamping and simultaneous contralateral nephrectomy (unilateral IRI-AKI). In a separate cohort, mice were treated with vehicle or 200 μg of PHAD followed by bilateral IRI-AKI. Mice were monitored for 3 days after reperfusion and euthanized.

Kidney function was assessed by blood urea nitrogen (BUN) and serum creatinine measurements. Kidney tubular injury was assessed by semi-quantitative analysis of tubular morphology on PAS-stained kidney sections, and kidney mRNA quantification of injury (N-Gal, Kim-1 and HO-1) and inflammation (IL-6, IL-1b and TNF-α) using qRT-PCR technique. Additional studies were conducted using immunohistochemistry to quantify Kim-1 and F4/80 protein to assess kidney injury and macrophages, respectively. Pretreatment with PHAD followed by unilateral IRI-AKI yielded a dose-dependent preservation of renal function. Histological injury and N-Gal mRNA were lower, and IL-1b mRNA higher in 200 μg 3D 6-Acyl PHAD treated mice. Similar protective effects of pretreatment with PHAD at 200 mg were seen after bilateral IRI-AKI, and there was significantly reduced Kim-1 immunostaining in the outer medulla of mice treated with PHAD after bilateral IRI-AKI.

PHAD-induced trained immunity leads to robust, dose-dependent protection from renal injury after unilateral and bilateral IRI-AKI in mice.

### Example 2: Pretreatment with a Novel Toll-Like Receptor 4 Agonist Attenuates Renal Ischemia-Reperfusion Injury

One strategy for preventing perioperative AKI that shows promise in this field is pretreatment with toll-like receptor 4 (TLR4) agonists, which have been shown to protect against organ damage associated with cardiac and striated muscle injury, neuronal injury, and sepsis, but have not been evaluated after ischemia reperfusion-induced acute kidney injury (IRI-AKI). Trained immunity is induced upon priming innate leukocytes with pathogen-derived products. This confers innate immune memory, yielding long-term and broad-spectrum resistance to infection. The toll-like receptor 4 ligands, lipopolysaccharide (LPS)- or monophosphoryl lipid A (MPLA), induce innate immune memory and lead to a controlled inflammatory cytokine response during the development of a clinically relevant severe infection. However, LPS is toxic to humans primarily due to the two phosphate groups and MPLA is no longer available for pharmaceutical development outside of its role as a vaccine adjuvant. Three novel, ultrapure synthetic TLR4 agonists, belonging to the phosphorylated hexa-acyl disaccharides class, have been shown to induce innate immune broad-spectrum antimicrobial functions for up to 10 days, and a regulated cytokine response, but without the toxicity of LPS. Unlike MPLA, all three phosphorylated hexa-acyl disaccharides are candidates for pharmaceutical development. Herein, it is contemplated and tested that pretreatment with the phosphorylated hexa-acyl disaccharide, monophosphoryl Hexa-acyl Lipid A, 3-Deacyl 6-acyl phosphorylated hexa-acyl disaccharide (PHAD) confers organ protection in mouse models of IRI-AKI. To one's knowledge, these studies are the first to characterize PHAD-induced organ protection in models of IRI-AKI.

### Materials and Methods

Mice. Studies were approved by the Institutional Animal Care and Use Committee at Vanderbilt University Medical Center (Protocol #M1800068-01) and complied with the National Institutes of Health Guide for the Care and Use of Experimental Animals. In accordance with the National Research Council's Guide for the Care and Use of Laboratory Animals and the Public Health Service Policy on Humane Care and Use of Laboratory Animals, mice were housed in an American Association for Accreditation of Laboratory Animal Care-accredited animal facility. Specifically, mice had continuous access to laboratory rodent diet (LabDiet®) and water, and lights were maintained on from 6 am through 9 pm.

Ischemia Reperfusion Induced Acute Kidney Injury. Male BALB/c mice, 10- to 11-weeks old (22 to 25 g) were purchased from Charles River (Hollister, CA), and were allowed to acclimate for one week prior to studies. IRI-AKI surgery was performed under ketamine/xylazine anesthesia (120 mg/kg [3 mg ketamine], 12 mg/kg [0.3 mg] xylazine) administered in 100 μl sterile saline by intraperitoneal (IP) injection. Mice underwent two types of surgery to induce IRI-AKI. Unilateral IRI-AKI with contralateral nephrectomy (unilateral IRI), and in separate cohorts, bilateral IRI-AKI. For unilateral IRI, the mouse was placed prone on a heat pad set to 37° C., and incisions were made in the muscle and skin to exteriorize the right kidney for nephrectomy. The adrenal gland was displaced from the kidney, and 5-0 silk suture (Ethicon, LA53G) was tied around the renal artery, vein, and ureter with a double surgical knot, before the kidney was removed by cutting distally to the knot. The left kidney was then exteriorized, and fat and connective tissue were dissected from the renal pedicle to expose the renal artery and vein. A vascular clamp (Roboz, cat #RS-5459) was applied to the renal vasculature for 28 minutes. The kidney was tucked back under the skin and the mouse was kept warm on the heat pad during the 28 minutes of ischemia time. For bilateral IRI-AKI, mice underwent bilateral renal artery ischemia for 24 minutes without nephrectomy. Successful ischemia was confirmed by a dark purple appearance of the kidney prior to clamp removal, and successful reperfusion was confirmed by a return to the normal pink color of the kidney within 1-2 min of clamp removal. Upon removing the vascular clamp, the muscle wall was closed with 6-0 absorbable suture (Ethicon, J492G) followed by skin closure with 7 mm wound clips (Roboz, RS-9255, RS-9250). Upon skin closure, mice received 0.5 ml of warm sterile saline subcutaneous (SQ) immediately after surgery, and at 24 hours after surgery. Analgesia was provided with buprenorphine SQ immediately after surgery and every 12 hours for 3 days. Sham operated mice underwent the same surgical approaches (for unilateral IRI-AKI, they underwent right nephrectomy, but the left renal pedicle was not clamped, and for bilateral IRI-AKI, both kidneys were exteriorized but the renal pedicles were not clamped). Blood was collected at baseline (prior to injury), and days 1 and 3 after injury by submandibular vein puncture into a lithium-heparin coated microcuvette tubes, and plasma frozen at −80° C. for blood urea nitrogen (BUN) and creatinine assays. On day 3 post-IRI, mice underwent terminal anesthesia using 5% isoflurane, and upon achieving a respiratory rate of 5 breaths per minute, a laparotomy and sternotomy were performed, and the terminal day 3 blood samples were collected by direct left ventricular apical cardiac puncture followed by cervical dislocation. BUN levels were measured by adding 200 μl Infinity Urea reagent (Thermo Scientific TR12421) to 2 l plasma or urea standard (Pointe Scientific B7550-STD) in duplicate in a 96-well plate (Thermo Scientific 130188). Absorbance was measured at 340 nm and 405 nm and urea nitrogen was calculated as described by the manufacturer. Serum creatinine was assessed by liquid chromatography mass spectrometry (LC-MS) at the O'Brien Core Center for Acute Kidney Injury Research at the University of Alabama at Birmingham.

Treatment with monophosphoryl hexa-acyl Lipid A, 3-Deacyl 6-acyl phosphorylated hexa-acyl disaccharide (PHAD). PHAD was synthesized de novo by Avanti Polar Lipids (Lot: 699852P-1MG-B-012; Alabaster, AL), dissolved in endotoxin-free water containing 0.2% trimethylamine and sonicated in a 40° C. water bath for 1 hour. Dilutions of PHAD were prepared for intravenous (IV) injection using Lactated Ringers (LR) solution. Intravenous priming was performed via a penile vein injection using vehicle or PHAD at 2, 20 or 200 g/0.2 mL per mouse. Vehicle or PHAD were administered at 48 and 24 hours prior to IRI-AKI.

Randomization and Blinding. Mice were randomized into 5 groups: Sham, vehicle treated, and treatment with either IV (N=10) 2, 20 or 200 g of PHAD per mouse. Randomization was accomplished using a website random team generator (Random Lists; www.randomlists.com/team-generator). Cages were not randomized, but rather individual mice were randomized upon opening each cage, according to the randomization table generated by the website. The randomizer assigns mice of each group to each cage. The first mouse of cage 1 was selected at random and marked according to first assigned mouse on the randomization table, and the second mouse was selected at random and marked according to the second assigned mouse on the randomization table, and so on and so forth. This allowed us to control for any differences that may exist between cages, although these cages were located immediately adjacent to each other to control for light cycle and animal care handlers. Scientists injecting the mice, performing the surgeries, and conducting the data analyses were blinded as to the treatment groups until each of the analyses had been completed (including BUN, QRT-PCR, histology and immunostaining studies).

Histological analysis of tubular injury. Upon euthanasia, the right atrium was cannulated with a 22 g needle, and perfusion was performed with 10 ml of 1×PBS. The right kidney pedicle was ligated, and kidney was excised. The 22 g needle was kept in situ, the left kidney was perfused using 10 ml of 10% formalin fixation solution. The left kidney was excised and cut along the transverse plane and two 2 mm sections were excised from the center of each kidney half, one section was minced and stored at −80° C. and the other was immediately immersed in 10% formalin for four hours. Upon completing the 4-hour 10% formalin fixation, the tissue was washed thrice in PBS, then submersed in 70% ethanol overnight at 4° C. The following day kidney sections were processed and embedded in paraffin using standard histological techniques. 5 m sections were deparaffinized and hydrated by standard methods and were stained with PAS using a PAS staining kit (Sigma, 395B-1KT) as described by the manufacturer. Semiquantitative histologic scoring of the outer stripe of the outer medulla of each section was performed on a 5-point severity scale of tubular degeneration/necrosis where 0=within normal limits, 1=minimal, 2=mild, 3=moderate, 4=marked, 5=severe. An aggregate score for the whole region of interest was given for each section by a pathologist blinded to the treatment group (L.H.).

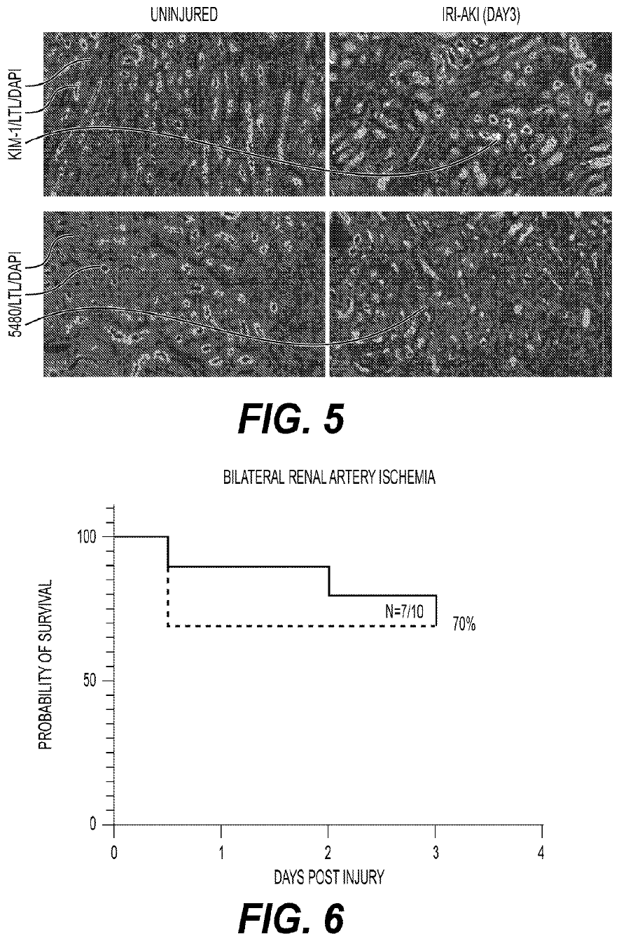

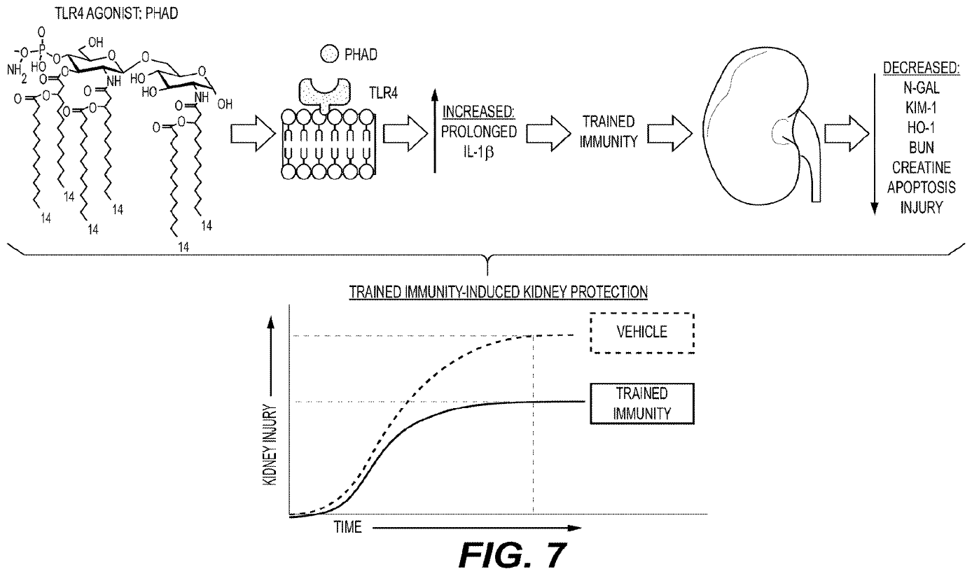

Immunohistochemistry staining and quantification. The control group animals served as internal baseline for the purpose of this study. Immunofluorescence controls were performed during optimization of the immunofluorescence antibodies. Formalin-fixed, paraffin-embedded kidney sections were deparaffinized and rehydrated in preparation for immunofluorescence labeling. Slides were heated in a steamer for 26 minutes in Target Retrieval Solution, Citrate pH 6.1 (Agilent, S169984-2). They were washed and then blocked with an Avidin/Biotin Blocking kit (Vector Labs, H-4000) and Power Block (BioGenex, HK085) diluted in PBS for 30 minutes prior to incubation with primary antibodies. Each section was incubated overnight at 4° C. with LTL-biotin (Vector Labs, B-1325), rat anti-F4/80 monoclonal antibody diluted to 1/250 [CI:A3-1](abcam, ab6640), or rat anti-Kim-1 monoclonal antibody (R&D Systems, MAB1817), diluted to 1/500 in 2.5% BSA and 0.05% Tween20 in PBS. The following day, slides were washed and incubated with NeutrAvidin Protein, DyLight 488 (Thermo Fisher, 84607) and Cy5 AffiniPure Donkey Anti-Rat IgG (H+L) (Jackson Immunoresearch, 712-175-153) diluted to 1/500 in 2.5% BSA and 0.05% Tween20 in PBS. Sections were thoroughly washed and incubated overnight with directly conjugated antibody. The nuclei were labeled using Hoechst 33342 (Thermo Fisher, 62249) diluted to 1:5000 in PBS. Slides were mounted in ProLong Gold Antifade Mountant (Thermo Fisher, P10144) and air dried overnight. Digital images were acquired by scanning the stained slides using a Zeiss AxioScan Z1. Co-labeling of LTL with the anti-Kim-1 monoclonal antibody under these conditions shows correct localization of Kim-1 limited to LTL+ proximal tubules after IRI-AKI but lack of staining in uninjured kidneys, and while the F4/80 monoclonal antibody shows correct F4/80 immunostaining limited to the interstitial space surrounding tubules after IRI-AKI, with only sparse interstitial staining in uninjured kidneys (FIG. 5).

Quantitative analysis of digital images was completed under blinded conditions using QuPath v0.3.0. Regions of interest were manually annotated using LTL staining and the arcuate vessels as landmarks, enabling signals in the cortex and outer stripe of the outer medulla (OSOM) to be quantified separately. Areas of immunopositivity per region per kidney were recorded for Kim-1 and F4/80, and results displayed as mean+/−SEM, and individual datapoints are shown.

TUNEL staining and analysis. Formalin-fixed, paraffin-embedded kidney sections were deparaffinized and rehydrated in preparation for TUNEL staining. Slides were placed on the Leica Bond RX IHC Stainer. All steps besides dehydration, clearing and cover-slipping are performed on the Bond RX. Slides were deparaffinized. Antigen retrieval was performed on the Bond RX using Triton X-100 (Cat #T9284, St. Louis, MO) for 5 minutes. Slides were then incubated with Equilibration Buffer (#G7130, Promega, Madison, WI) for 5 minutes, followed with the TdT reaction mix (#G7130, Promega, Madison, WI) for 10 minutes, and SSC-x20 (#G7130, Promega, Madison, WI) for 10 minutes. The slides were incubated with Streptavidin-HRP (#RE7104, Novocastra, Newcastle Upon Tyne, United Kingdom) for 5 minutes, and the Bond Refine detection system (#DS9800, Leica, Buffalo Grove, IL) was used for visualization. After staining, sections were counterstained using hematoxylin (#7211, Epredia), as outlined in the manufacturer's instruction, dehydrated, cleared and cover-slipped. Digital images were acquired by scanning the stained slides using a Zeiss AxioScan Z1, and quantitative analysis of digital images was completed under blinded conditions using QuPath v0.3.0. The outer stripe of the outer medulla was manually annotated using the arcuate vessels as outer and S3 segment proximal tubules as inner landmarks. All tubules were selected and were classified as having viable or containing necrotic cellular casts. Once necrotic cast-filled tubules were excluded, a positive cell detector was run within QuPath to detect all nuclei (counterstained using hematoxylin), and DAB (TUNEL) positive nuclei using the same threshold for all of the stained sections. The output data identified all positively stained nuclei in the defined areas and results expressed as the percentage TUNEL positive/total nuclei in the outer stripe of the outer medulla. Masked areas containing DAB positive necrotic casts were excluded.

Quantitative real-time PCR. A central transverse kidney section was lysed in a Lysing Matrix A tube (MP Biomedicals, Irvine, CA, USA) using a MiniBeater-16 (BioSpec Products, Bartiesville, OK, USA) and RNA was isolated using a RNeasy Mini kit (Qiagen, Hilden, Germany). cDNA was synthesized from 1 g RNA using iScript cDNA synthesis kit (Bio-Rad, Hercules, CA, USA). Quantitative mRNA expression was determined by real time PCR using iQ SYBR Green supermix (Bio-Rad) with the following pair of primers (Table 1). Real-time PCR was performed in duplicate on a CFX96 Touch Real-Time PCR Detection System (Bio-Rad), and quantification of gene expression was determined by delta-delta cycle threshold (ΔCt) method, normalized for Gapdh mRNA expression.

Statistics. All data were analyzed using GraphPad Prism software version 9.3.1 (GraphPad Software, San Diego, CA, USA). Between group analyses were performed by Mann Whitney U test, to compare two groups, and from multiple group experiments by non-parametric one-way ANOVA (Kruskal Wallis test), for between group testing, using Dunnett's post hoc correction for multiple between group comparison. A two-way ANOVA was used to compare groups followed over time followed by Sidak's post hoc correction for multiple between group comparisons, and survival curves for were compared by Log-rank test. A value of p<0.05 was considered statistically significant.

### Results

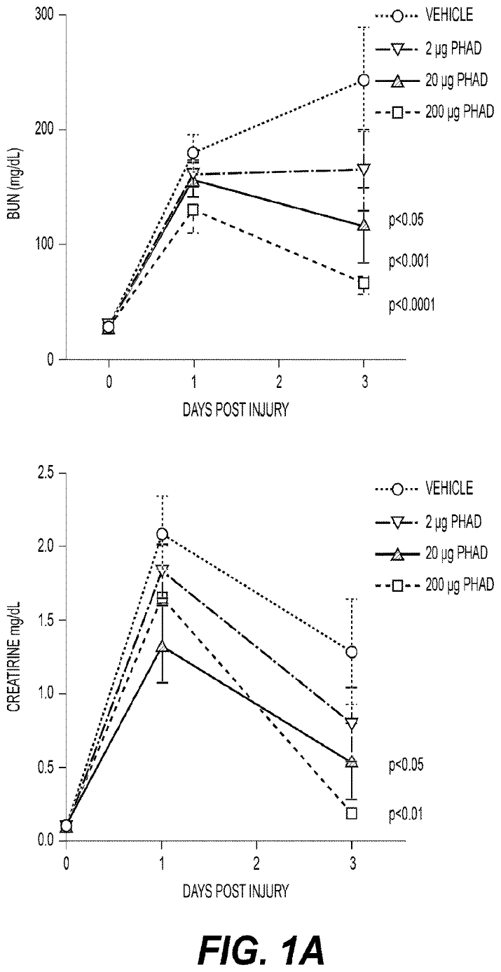

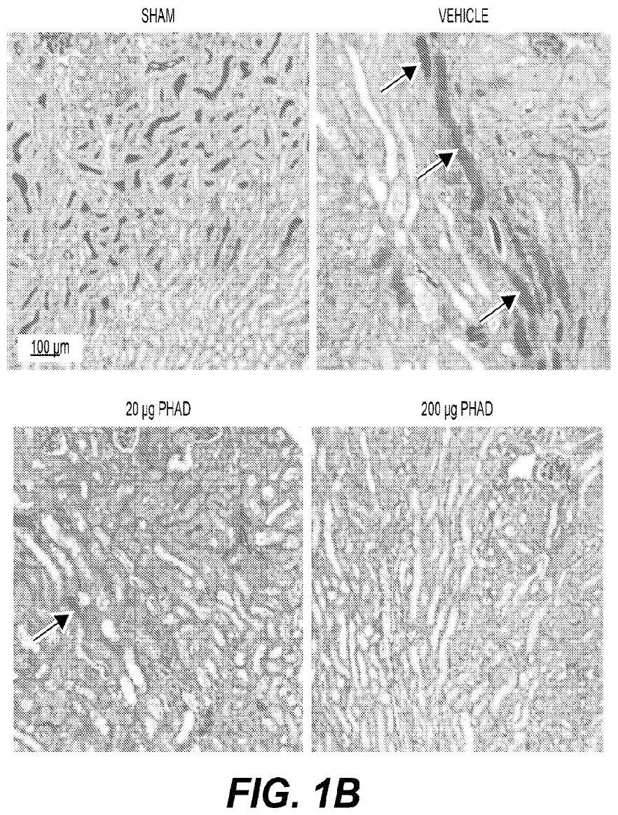

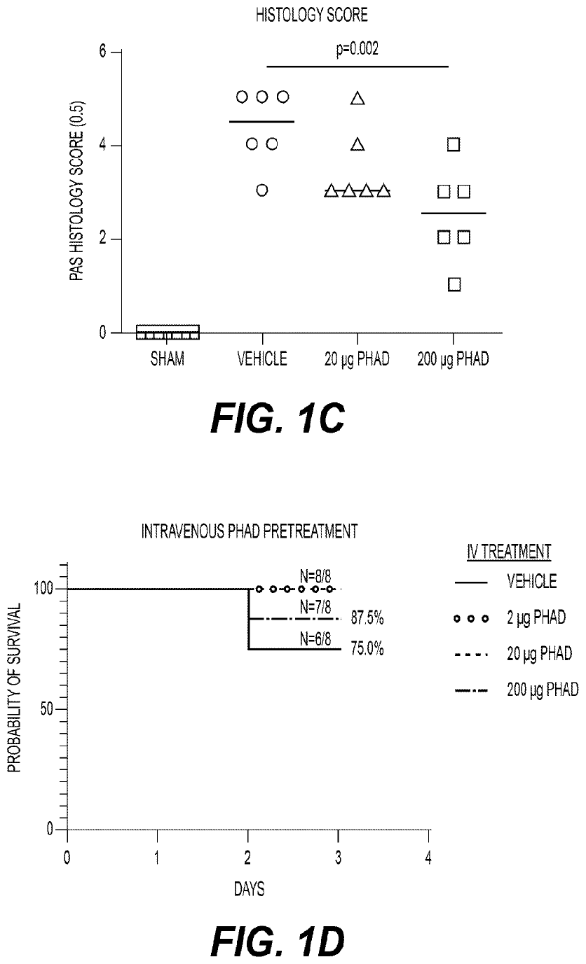

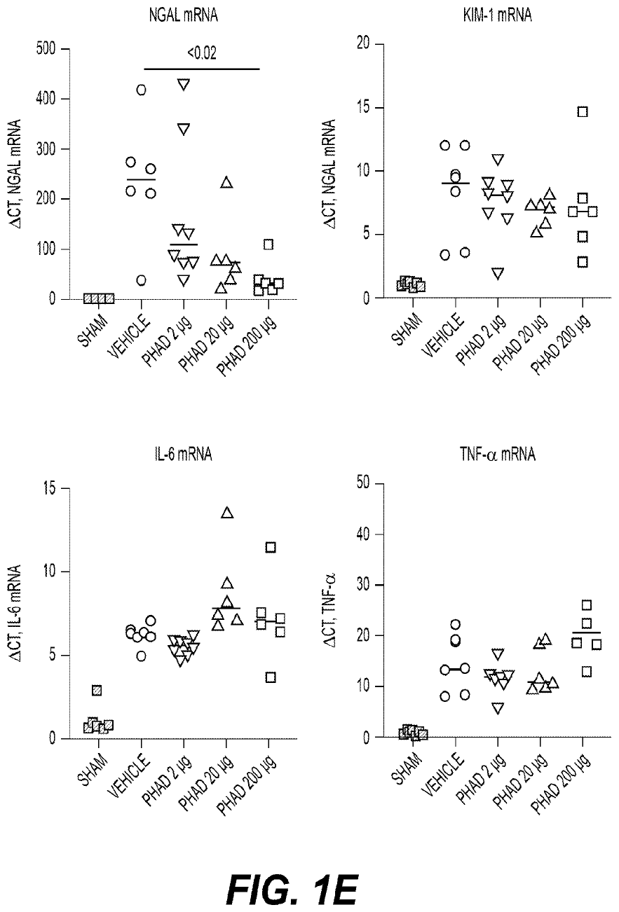

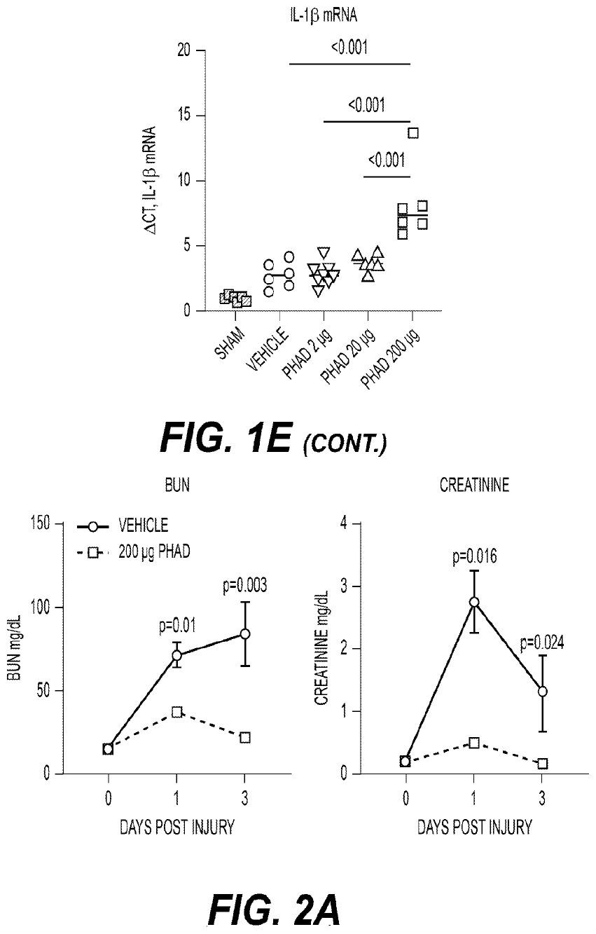

PHAD pretreatment preserves renal function and reduces renal injury after unilateral IRI-AKI. There was a dose-dependent decrease in IRI-induced kidney injury, as determined by reduced elevations in BUN and creatinine levels, in mice pretreated with IV PHAD (FIG. 1A). Intravenous PHAD treatment also reduced tubular injury in the cortex and OSOM, with reduced interstitial edema, and fewer casts with increasing doses of PHAD (FIGS. 1B and 1C). There was no significant improvement in survival in PHAD treated mice, but more mice survived at the 2 and 20 μg pretreatment dose compared with the vehicle control group (FIG. 1D). In addition, there was a reduction in expression of distal tubular injury marker N-Gal but no reduction in the proximal tubular injury marker Kim-1 mRNA in mice treated with 200 μg PHAD (FIG. 1E). This was associated with a dose-dependent increase in IL-1β but not Tnf-α or IL-6 mRNAs compared with vehicle control mice (FIG. 1E).

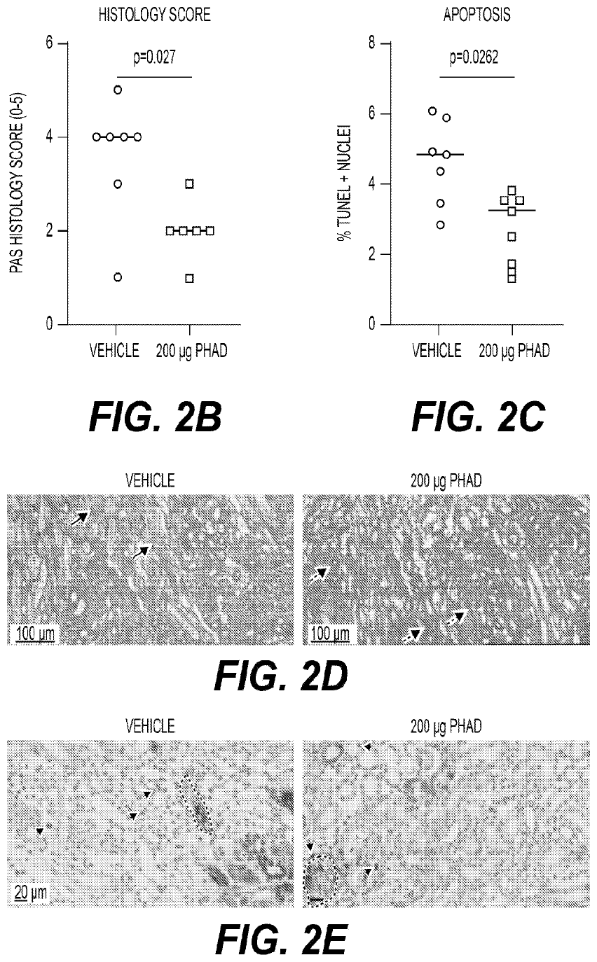

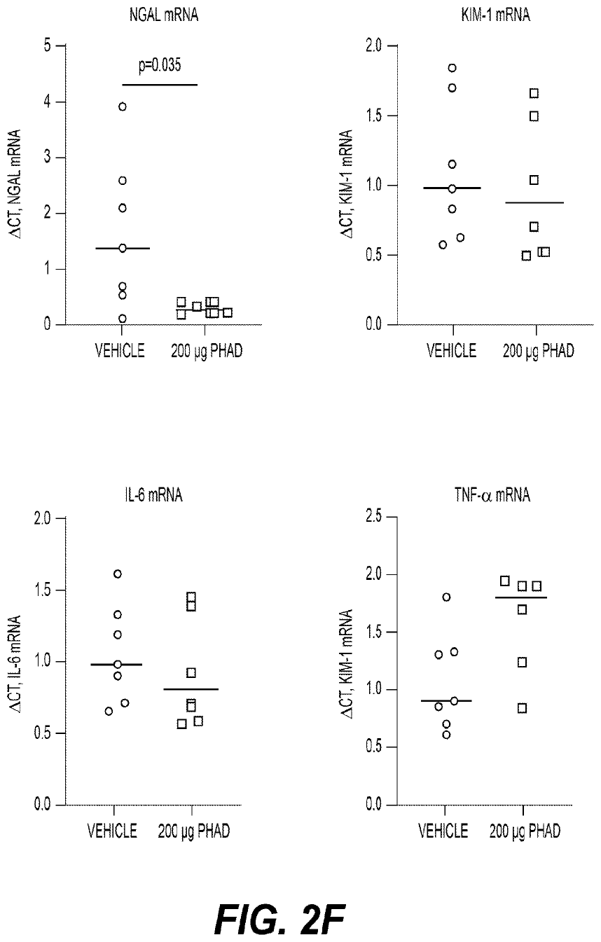

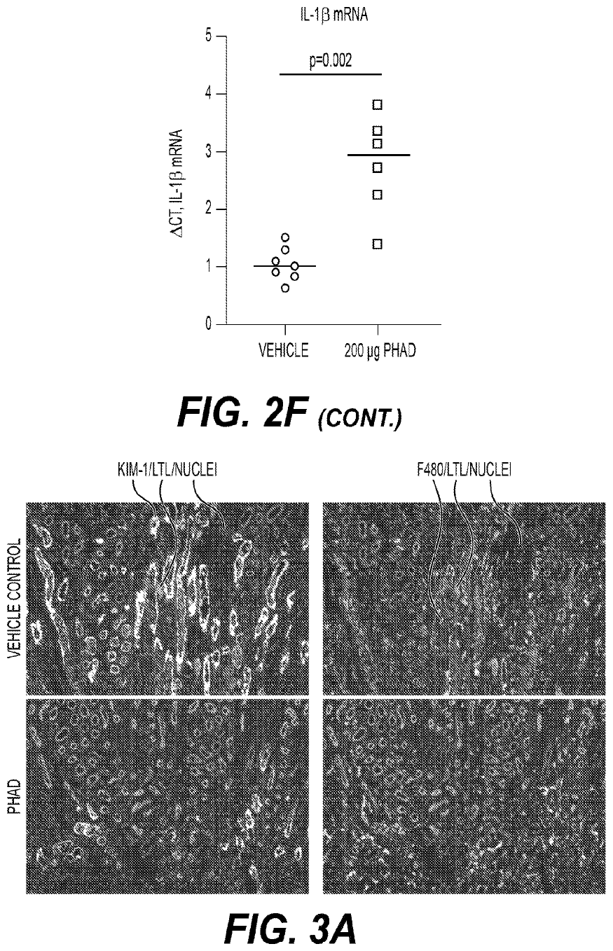

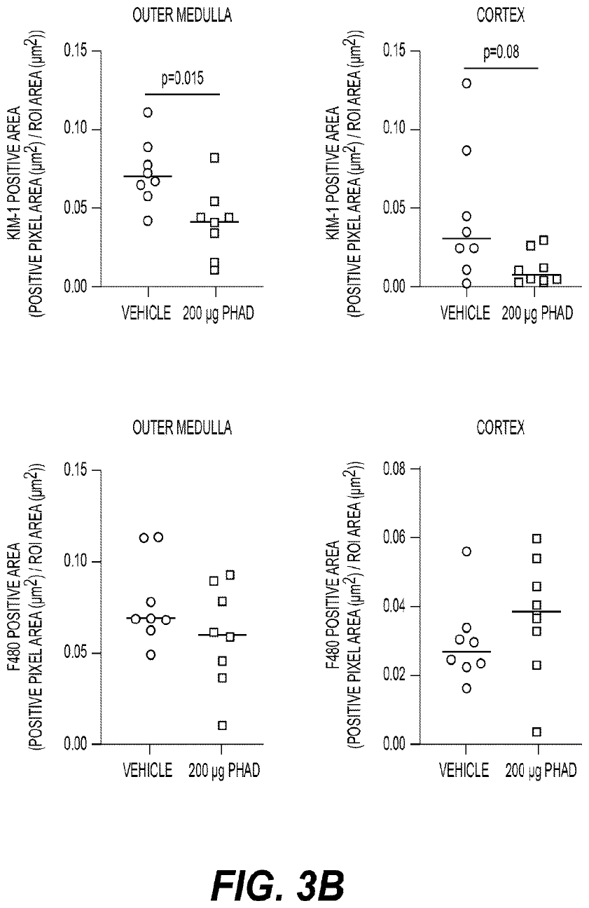

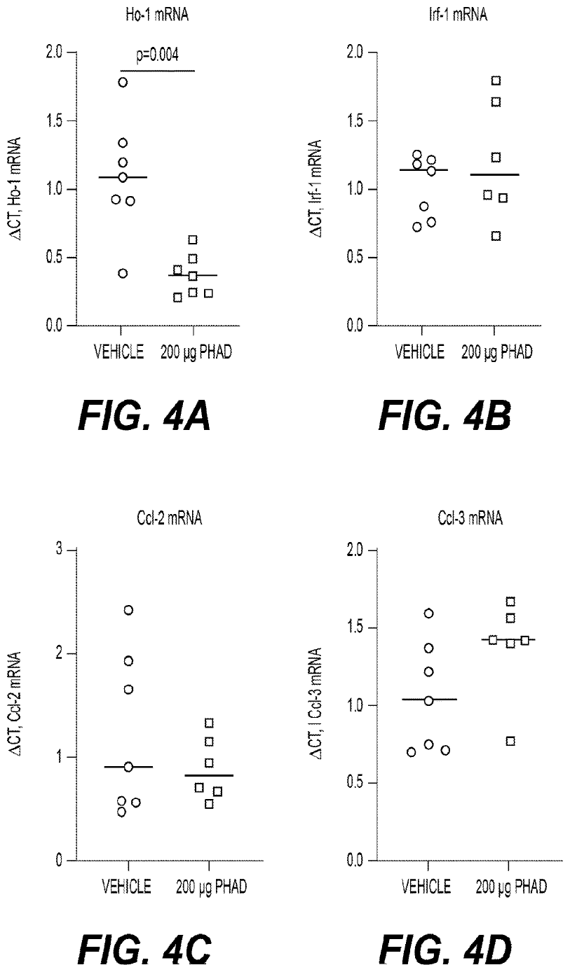

PHAD preserves renal function and reduces renal injury after bilateral IRI-AKI. The effects of pretreatment with PHAD at 200 μg/mouse was evaluated on renal function and tissue injury after bilateral IRI-AKI. Bilateral IRI-AKI has different pathophysiology compared to unilateral IRI-AKI since removal of the contralateral kidney in unilateral IRI-AKI increases blood flow to the remaining injured kidney, accelerating recovery compared with bilateral IRI-AKI. Renal injury was markedly reduced, as assessed by reduced BUN and creatinine levels, at 24 and 72 hours after bilateral IRI-AKI in IV PHAD treated mice (FIG. 2A), but there was no difference in survival between group (FIG. 6). PHAD treatment also reduced tubular injury, with less interstitial edema and fewer tubular casts (FIGS. 2B and 2D), and reduced apoptosis (FIGS. 2C and 2E). As with unilateral IRI-AKI, there were lower levels of N-Gal and increased IL-1β mRNAs in PHAD treated compared with vehicle treated mouse kidneys, and no differences in renal Kim-1, IL-6, or Tnf-α mRNAs (FIG. 2F). There was, however, a significant reduction in the area of cells expressing Kim-1 protein in the outer medulla but not the cortex of mice treated with PHAD, indicating that there is a dissociation between Kim-1 mRNA and protein expression in these studies, but no difference in F4/80 staining for renal macrophages (FIG. 3). Consistent with these findings, renal expression of Ho-1 mRNA, a ubiquitous injury response marker that is induced in the kidney after injury, was also reduced in PHAD-treated mice (FIG. 4A). Irf-2, Ccl-2, and Ccl-3 were also examined given their roles in mediating inflammation-induced macrophage recruitment and polarization, and polymorphonuclear leukocyte recruitment, but saw no differences in expression of the inflammatory markers Irf-1, Ccl-2, or Ccl-3 mRNAs (FIGS. 4B, 4C, and 4D).

### Discussion

The present example demonstrates for the first time that pretreatment with the TLR4 agonist, PHAD, improves renal function and reduces renal tubular injury in two different mouse models of IRI-AKI. The two IRI-AKI models have different pathophysiologies since removal of the contralateral kidney in unilateral IRI-AKI increases blood flow to the remaining injured kidney, accelerating recovery compared with bilateral IRI-AKI. Pretreatment with IV PHAD provided clear, dose-dependent organ protection from unilateral IRI-AKI. Upon identifying the optimal dose of PHAD for conferring protection against unilateral IRI-AKI, in a separate cohort of mice this was tested after bilateral IRI-AKI and organ protection was observed. These results demonstrate that PHAD pretreatment provides equipotent organ protection in two models of perioperative AKI. While the translational significance of these two different models for human AKI are unclear, by showing a beneficial effect of PHAD pre-treatment in two models of IRI-AKI with somewhat different pathophysiologies, these findings indicate that the beneficial effects of PHAD are more robust than if only effective in one of the models.

Herein, PHAD was noted to reduce expression of the distal tubular injury marker, N-Gal, after IRI-AKI. No statistically significant differences were observed in Kim-1 mRNA expression, but a reduction in the surface area of cells staining with Kim-1 was observed in PHAD treated mice after bilateral IRI-AKI. The decrease in Kim-1 staining was paralleled by reduction in histological evidence of proximal tubular injury, and apoptosis, and reduced expression of Ho-1 mRNA, a ubiquitous injury response marker that is induced in the kidney after injury. This dissociation between Kim-1 mRNA and Kim-1 protein in these studies is either because the mRNA is maximally induced in both vehicle and PHAD treated mice, or has occurred for technical reasons because samples used for mRNA preparations have equal representation of cortex vs. outer medulla.

Previous efforts have shown that pretreatment with TLR4 agonists reprograms macrophage function to facilitate the development of trained immunity, which protects against infection and inflammation-induced injury (Fensterheim B A, Young J D, Luan L, Kleinbard R R, Stothers C L, Patil N K, McAtee-Pereira A G, Guo Y, Trenary I, Hernandez A, Fults J B, Williams D L, Sherwood E R, and Bohannon J K. The TLR4 Agonist Monophosphoryl Lipid A Drives Broad Resistance to Infection via Dynamic Reprogramming of Macrophage Metabolism. J Immunol 200: 3777-3789, 2018). IL-1β has been shown to directly induce trained immunity in human monocytes via epigenetic reprogramming mechanisms, and IL-1β also enhances glycolysis in hematopoietic progenitor cells. Consistent with these findings, a significant dose-dependent increase in expression of IL-1β mRNA was observed. In contrast to prior studies showing that pretreatment with LPS (a classical TLR4 agonist) before renal IRI, causes a sustained increase in renal Tnf-α or IL-6 mRNA in PHAD treated mice. These findings show that treatment with PHAD induces a controlled cytokine response, which is regulated and sustained upon the ischemic insult and is associated with renal protection after IRI. While changes in renal macrophage numbers were not observed by F4/80 staining in PHAD treated mice, macrophage function is contemplated to be altered in PHAD treated mice.

The findings herein indicate that intravenous administration of PHAD before injury provides marked, dose-dependent protective effects in two mouse models of IRI-AKI.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLES TABLE 1
Primers

| Primer | Forward sequence | Reverse sequence |
|---|---|---|
| Gapdh | TGGAGAAACCTGCCAAGTATGA | GAAGAGTGGGAGTTGCTGTTGA |
| | SEQ ID NO. 1 | SEQ ID NO. 2 |
| N-Gal | GCAGGTGGTACGTTGTGGG | CTCTTGTAGCTCATAGATGGTGC |
| | SEQ ID NO. 3 | SEQ ID NO. 4 |
| Kim-1 | ACAAACCAGACTGGAATGGC | GTCCACAAGGAGCAGTAGCA |
| | SEQ ID NO. 5 | SEQ ID NO. 6 |
| IL-6 | CTTCCATCCAGTTGCCTTCTTG | AATTAAGCCTCCGACTTGTGAAG |
| | SEQ ID NO. 7 | SEQ ID NO. 8 |
| Tnf-α | CGGAGTCCGGGCAGGT | CGGAGTCCGGGCAGGT |
| | SEQ ID NO. 9 | SEQ ID NO. 10 |
| IL-1β | GCAACTGTTCCTGAACTCAACT | ATCTTTTGGGGTCCGTCAACT |
| | SEQ ID NO. 11 | SEQ ID NO. 12 |
| Irf-1 | ATGCCAATCACTCGAATGCG | TTGTATCGGCCTGTGTGAATG |
| | SEQ ID NO. 13 | SEQ ID NO. 14 |
| Ccl2 | TGCATCTGCCCTAAGGTCTTC | AAGTGCTTGAGGTGGTTGTGG |
| | SEQ ID NO. 15 | SEQ ID NO. 16 |
| Ccl3 | TGCCCTTGCTGTTCTTCTCT | GATGAATTGGCGTGGAATCT |
| | SEQ ID NO. 17 | SEQ ID NO. 18 |
| Ho-1 | ACAGAGGAACACAAAGACCAG | GTGTCTGGGATGAGCTAGTG |
| | SEQ ID NO. 19 | SEQ ID NO. 20 |

SEQUENCES

```
  1. SEQ ID NO: 1-Gapdh Forward Primer
TGGAGAAACCTGCCAAGTATGA
  2. SEQ ID NO: 2-Gapdh Reverse Primer
GAAGAGTGGGAGTTGCTGTTGA
  3. SEQ ID NO: 3-N-Gal Forward Primer
GCAGGTGGTACGTTGTGGG
  4. SEQ ID NO: 4-N-Gal Reverse Primer
CTCTTGTAGCTCATAGATGGTGC
  5. SEQ ID NO: 5-Kim-1 Forward Primer
ACAAACCAGACTGGAATGGC
  6. SEQ ID NO: 6-Kim-1 Reverse Primer
GTCCACAAGGAGCAGTAGCA
  7. SEQ ID NO: 7-IL-6 Forward Primer
CTTCCATCCAGTTGCCTTCTTG
  8. SEQ ID NO: 8-IL-6 Reverse Primer
AATTAAGCCTCCGACTTGTGAAG
  9. SEQ ID NO: 9-Tnf-alpha Forward Primer
CGGAGTCCGGGCAGGT
10. SEQ ID NO: 10-Tnf-alpha Reverse Primer
CGGAGTCCGGGCAGGT
11. SEQ ID NO: 11-IL-1beta Forward Primer
GCAACTGTTCCTGAACTCAACT
12. SEQ ID NO: 12-IL-1beta Reverse Primer
ATCTTTTGGGGTCCGTCAACT
13. SEQ ID NO: 13-Irf-1 Forward Primer
ATGCCAATCACTCGAATGCG
14. SEQ ID NO: 14-Irf-1 Reverse Primer
TTGTATCGGCCTGTGTGAATG
15. SEQ ID NO: 15-Ccl2 Forward Primer
TGCATCTGCCCTAAGGTCTTC
16. SEQ ID NO: 16-Ccl2 Reverse Primer
AAGTGCTTGAGGTGGTTGTGG
17. SEQ ID NO: 17-Ccl3 Forward Primer
TGCCCTTGCTGTTCTTCTCT
18. SEQ ID NO: 18-Ccl3 Reverse Primer
GATGAATTGGCGTGGAATCT
19. SEQ ID NO: 19-Ho-1 Forward Primer
ACAGAGGAACACAAAGACCAG
20. SEQ ID NO: 20-Ho-1 Reverse Primer
GTGTCTGGGATGAGCTAGTG
```

1. A method of treating or preventing an acute kidney injury (AKI), the method comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

2. The method of claim 1, wherein the compound is a phosphorylated hexa-acyl disaccharide (PHAD).

3. The method of claim 1, wherein the compound is a 3-deacyl phosphorylated hexa-acyl disaccharide (3D-PHAD).

4. The method of claim 1, wherein the compound is a 3-D (6-acyl) phosphorylated hexa-acyl disaccharide (3D(6-acyl)PHAD).

5. The method of claim 1, wherein the compound is a synthetic toll-like receptor 4 (TLR4) agonist.

6. The method of claim 1, wherein the compound is administered intravenously.

7. The method of claim 1, wherein the compound is administered at a dose between 0.5 mg/kg-40 mg/kg.

8. The method of claim 1, wherein the compound is administered about 24 hours or less prior to a surgical procedure.

9. The method of claim 1, wherein the compound protects one or both kidneys from damage during the surgical procedure.

10. The method of claim 8, wherein the surgical procedure is selected from the group consisting of a cardiac surgery, a vascular surgery, a neurosurgery, a genitourinary surgery, an abdominal surgery, and a kidney surgery.

11. The method of claim 1, wherein the acute kidney injury is an ischemia reperfusion-induced acute kidney injury (IRI-AKI).

12. The method of claim 1, wherein the compound is further administered with an antibiotic, a laxative, an anesthetic, a muscle relaxant, a sedative, or any combination thereof prior to the surgical procedure.

13. A method of preserving kidney function during a surgical procedure, the method comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

14. The method of claim 13, wherein the compound is a phosphorylated hexa-acyl disaccharide (PHAD).

15. The method of claim 13, wherein the compound is a 3-deacyl phosphorylated hexa-acyl disaccharide (3D-PHAD).

16. The method of claim 13, wherein the compound is a 3-D (6-acyl) phosphorylated hexa-acyl disaccharide (3D(6-acyl)PHAD).

17.-23. (canceled)

24. A method of treating or preventing an ischemic injury during a surgical procedure, the method comprising administering to a subject in need thereof a pharmaceutically effective amount of a compound selected from a phosphorylated hexa-acyl disaccharide (PHAD), a 3-deacyl phosphorylated hexa-acyl disaccharide (3D-PHAD), a 3-D (6-acyl) phosphorylated hexa-acyl disaccharide (3D(6-acyl)PHAD), or a pharmaceutically acceptable salt thereof.

25. The method of claim 24, wherein the compound is a phosphorylated hexa-acyl disaccharide (PHAD).

26. The method of claim 24, wherein the compound is a 3-deacyl phosphorylated hexa-acyl disaccharide (3D-PHAD).

27. The method of claim 24, wherein the compound is a 3-D (6-acyl) phosphorylated hexa-acyl disaccharide (3D(6-acyl)PHAD).

28.-36. (canceled)


SEQUENCE LISTING (20 sequences)

SEQ ID NO: 1 - DNA, 22 bp, synthetic construct
  misc_feature 1..22: note = Description of sequence: GAPDH Forward Primer
  source 1..22: mol_type = other DNA
tggagaaacctgccaagtatga

SEQ ID NO: 2 - DNA, 22 bp, synthetic construct
  misc_feature 1..22: note = Description of sequence: GAPDH Reverse Primer
  source 1..22: mol_type = other DNA
gaagagtgggagttgctgttga

SEQ ID NO: 3 - DNA, 19 bp, synthetic construct
  misc_feature 1..19: note = Description of sequence: N-Gal Forward Primer
  source 1..19: mol_type = other DNA
gcaggtggtacgttgtggg

SEQ ID NO: 4 - DNA, 23 bp, synthetic construct
  misc_feature 1..23: note = Description of sequence: N-Gal Reverse Primer
  source 1..23: mol_type = other DNA
ctcttgtagctcatagatggtgc

SEQ ID NO: 5 - DNA, 20 bp, synthetic construct
  misc_feature 1..20: note = Description of sequence: Kim-1 Forward Primer
  source 1..20: mol_type = other DNA
acaaaccagactggaatggc

SEQ ID NO: 6 - DNA, 20 bp, synthetic construct
  misc_feature 1..20: note = Description of sequence: Kim-1 Reverse Primer
  source 1..20: mol_type = other DNA
gtccacaaggagcagtagca

SEQ ID NO: 7 - DNA, 22 bp, synthetic construct
  misc_feature 1..22: note = Description of sequence: IL-6 Forward Primer
  source 1..22: mol_type = other DNA
cttccatccagttgccttcttg

SEQ ID NO: 8 - DNA, 23 bp, synthetic construct
  misc_feature 1..23: note = Description of sequence: IL-6 Reverse Primer
  source 1..23: mol_type = other DNA
aattaagcctccgacttgtgaag

SEQ ID NO: 9 - DNA, 16 bp, synthetic construct
  misc_feature 1..16: note = Description of sequence: Tnf-alpha Forward Primer
  source 1..16: mol_type = other DNA
cggagtccgggcaggt

SEQ ID NO: 10 - DNA, 16 bp, synthetic construct
  misc_feature 1..16: note = Description of sequence: Tnf-alpha Reverse Primer
  source 1..16: mol_type = other DNA
cggagtccgggcaggt

SEQ ID NO: 11 - DNA, 22 bp, synthetic construct
  misc_feature 1..22: note = Description of sequence: IL-1beta Forward Primer
  source 1..22: mol_type = other DNA
gcaactgttcctgaactcaact

SEQ ID NO: 12 - DNA, 21 bp, synthetic construct
  misc_feature 1..21: note = Description of sequence: IL-1beta Reverse Primer
  source 1..21: mol_type = other DNA
atcttttggggtccgtcaact

SEQ ID NO: 13 - DNA, 20 bp, synthetic construct
  misc_feature 1..20: note = Description of sequence: Irf-1 Forward Primer
  source 1..20: mol_type = other DNA
atgccaatcactcgaatgcg

SEQ ID NO: 14 - DNA, 21 bp, synthetic construct
  misc_feature 1..21: note = Description of sequence: Irf-1 Reverse Primer
  source 1..21: mol_type = other DNA
ttgtatcggcctgtgtgaatg

SEQ ID NO: 15 - DNA, 21 bp, synthetic construct
  misc_feature 1..21: note = Description of sequence: Ccl2 Forward Primer
  source 1..21: mol_type = other DNA
tgcatctgccctaaggtcttc

SEQ ID NO: 16 - DNA, 21 bp, synthetic construct
  misc_feature 1..21: note = Description of sequence: Ccl2 Reverse Primer
  source 1..21: mol_type = other DNA
aagtgcttgaggtggttgtgg

SEQ ID NO: 17 - DNA, 20 bp, synthetic construct
  misc_feature 1..20: note = Description of sequence: Ccl3 Forward Primer
  source 1..20: mol_type = other DNA
tgcccttgctgttcttctct

SEQ ID NO: 18 - DNA, 20 bp, synthetic construct
  misc_feature 1..20: note = Description of sequence: Ccl3 Reverse Primer
  source 1..20: mol_type = other DNA
gatgaattggcgtggaatct

SEQ ID NO: 19 - DNA, 21 bp, synthetic construct
  misc_feature 1..21: note = Description of sequence: Ho-1 Forward Primer
  source 1..21: mol_type = other DNA
acagaggaacacaaagaccag

SEQ ID NO: 20 - DNA, 20 bp, synthetic construct
  misc_feature 1..20: note = Description of sequence: Ho-1 Reverse Primer
  source 1..20: mol_type = other DNA
gtgtctgggatgagctagtg